(19) United States
(12) Patent Application Publication
(10) Pub. No.: US 2022/0301722 A1
(43) Pub. Date: Sep. 22, 2022

(54) DRIVE-THROUGH TREATMENT SYSTEM AND METHOD

(71) Applicants: Hyundai Motor Company, Seoul (KR); Kia Corporation, Seoul (KR)

(72) Inventor: Joon Young KIM, Seongnam-Si (KR)

(73) Assignees: Hyundai Motor Company, Seoul (KR); Kia Corporation, Seoul (KR)

(21) Appl. No.: 17/538,613

(22) Filed: Nov. 30, 2021

(30) Foreign Application Priority Data
Mar. 16, 2021 (KR) 10-2021-0033711

(51) Int. Cl. G16H 50/30; G06V 40/16; G16H 50/80; G16H 40/67

(52) U.S. Cl. CPC G16H 50/30; G06V 40/166; G06V 40/169; G06V 40/174; G16H 50/80; G16H 40/67

(57) ABSTRACT

A drive-through treatment system includes a monitoring device configured to generate a plurality of body information data by detecting a plurality of body information related to a service user in a vehicle, and to generate transit section data by acquiring GPS data related to a movement path of the vehicle from the vehicle when the vehicle enters a section where a drive-through treatment system is established; and a central management device configured to receive the plurality of body information data and the transit section data from the monitoring device, and to determine a condition of the service user according to the plurality of body information data and the transit section data.

### CROSS-REFERENCE TO RELATED APPLICATION

The present application claims priority to Korean Patent Application No. 10-2021-0033711, filed on Mar. 16, 2021, the entire contents of which is incorporated herein for all purposes by this reference.

### BACKGROUND OF THE INVENTION

### Field of the Invention

The present invention relates to a drive-through treatment system and a drive-through treatment method.

### Description of Related Art

In the case of the existing drive-through examination service, only simple information was delivered and limited examination was provided in the examination service. Even when service user information is delivered to medical staff, only the information written by a service user is delivered to the medical staff due to environmental constraints of the examination service. There are many cases in which the amount of information is not sufficient because the service user has to write it by hand. The present limited delivery of information increases a time required to provide a medical examination service.

Due to the limited information related to the service user, it is difficult for the medical staff to prepare and cope with it appropriately depending on a condition of the service user. Furthermore, when examination and treatment are performed without information related to the service user, not only the medical staff, but also many people who are positioned in a drive-through clinic may be exposed to a risk of infection.

The information disclosed in this Background of the Invention section is only for enhancement of understanding of the general background of the invention and may not be taken as an acknowledgement or any form of suggestion that this information forms the prior art already known to a person skilled in the art.

### BRIEF SUMMARY

Various aspects of the present invention are directed to providing a drive-through treatment system and method configured for reducing the time required to provide an examination service.

Furthermore, various aspects of the present invention are directed to providing a drive-through treatment system and method configured for providing services suitable for service users by delivering accurate information related to conditions of service users and blocking many people positioned in drive-through clinics, such as medical staffs, from a risk of infection.

Various aspects of the present invention are directed to providing a drive-through treatment system including: a monitoring device configured to generate a plurality of body information data by detecting a plurality of body information related to a service user in a vehicle, and to generate transit section data by acquiring Global Positioning System (GPS) data related to a movement path of the vehicle from the vehicle when the vehicle enters a section where a drive-through treatment system is established; and a central management device configured to receive the plurality of body information data and the transit section data from the monitoring device, and to determine a condition of the service user according to the plurality of body information data and the transit section data.

The drive-through treatment system may further include medical treatment devices provided in a plurality of medical shelters, and the central management device may transmit the condition of the service user to the medical treatment devices.

The monitoring device may include: a camera configured to generate facial image data by photographing a face of the service user; an infrared sensor (IR sensor) configured to detect a temperature of the service user to generate temperature data; a data converter electrically connected to the camera and the IR sensor and configured to receive the facial image data, the temperature data, and the GPS data, and to convert each of the received facial image data, temperature data and transit section data depending on a predetermined conversion method to data; a data filter unit configured to receive and filter the data received from the data converter to remove noise from the received data; and a verification unit configured to verify data received through the data filter unit based on a matching range corresponding to each of the data received through the data filter unit.

The data converter may determine main points for determining a facial contour and an expression based on the facial image data, and may generate facial feature data representing the main points.

The data converter may generate body temperature data by converting the temperature data depending on a body temperature range of a person.

The data converter may determine a movement path of a service user who has moved by the vehicle based on the GPS data, may determine sections through which the vehicle passes depending on the movement path, and may generate the transit section data indicating the sections through which the vehicle passes depending on an elapsed time taken by the vehicle.

The data received through the data filter unit may include the facial feature data, the body temperature data, and the transit section data. The verification unit may change each of the data to a corresponding set value when each of the received data deviates from a matching range corresponding to each of the facial feature data, the body temperature data, and the transit section data.

The central management device may include: a data augmenter configured to augment the body information data and the transit section data to generate augmented data; and a condition determination unit configured to receive the augmentation data from the data augmenter, and to determine a condition level by determining the condition of the service user according to the augmentation data.

The data augmenter may convert the body information data and the transit section data into input data in a form suitable for the condition determination unit.

The plurality of body information data may include facial feature data and body temperature data. The data augmenter may generate the augmented data by serially arranging the facial feature data, the body temperature data, and the transit section data.

The condition determination unit may include: a feature derivation unit configured to analyze the augmented data to determine a current pain degree of the service user, a body temperature of the service user, and a number of infectious disease occurrence sections that the service user has passed through; a parameter determination unit configured to determine parameters for the current pain degree of the service user, the body temperature of the service user, and the number of infectious disease occurrence sections that the service user has passed through; a level determination unit configured to determine a pain level, a body temperature level, and an infection risk level for the current pain degree of the service user, the body temperature of the service user, and the infectious disease occurrence sections that the service user has passed through; and an index mapping unit configured to determine a value obtained by multiplying each of the pain level, the body temperature level, and the infection risk level by a corresponding one of the parameters, and determine an index indicating the condition of the service user by adding the determined multiplied values.

The index mapping unit may compare the index with a plurality of reference values, and determines a condition level indicating the condition of the service user according to a result of the comparing.

The index mapping unit may determine the condition level as a value indicating a stable state when the index is smaller than a first reference, may determine the condition level as a value indicating a stable state that requires attention when the index is greater than or equal to the first reference value and smaller than a second reference value, may determine the condition level as a value indicating an unstable state that requires attention when the index is greater than or equal to the second reference value and smaller than a third reference value, may determine the condition level as a value indicating a urgent unstable state when the index is greater than or equal to the third reference value and smaller than a fourth reference value, and may determine the condition level as a value indicating an emergency unstable condition when the index is greater than or equal to the fourth reference value.

Various aspects of the present invention are directed to providing a drive-through treatment method including: detecting, by a monitoring device, a vehicle when the vehicle enters a section where a drive-through treatment system is established; generating, by the monitoring device, a plurality of body information data by detecting a plurality of body information related to a service user in the vehicle, and generating transit section data by acquiring Global Positioning System (GPS) data related to a movement path of the vehicle from the vehicle; and receiving, by a central management device, the plurality of body information data and the transit section data from the monitoring device, and determining a condition of the service user according to the plurality of body information data and the transit section data.

The drive-through treatment method may further include transmitting, by the central management device, a condition level indicating the condition of the service user to a plurality of medical treatment devices provided in a plurality of medical shelters.

The drive-through treatment method may further include displaying, by each of the medical treatment devices, the condition level received from the central management device through a display device.

The drive-through treatment method may further include: transmitting, by the monitoring device, a notification of failure to detect the body information to the central management device when the monitoring device fails to detect the body information related to the service user; and notifying the medical treatment devices provided in the medical shelters of the failure to detect the body information when the central management device receives the notification of the failure to detect the body information.

As described above, according to various exemplary embodiments of the present invention, a drive-through treatment system and method capable of reducing a time required to provide an examination service is provided.

Furthermore, it is possible to provide a drive-through treatment system and method capable of providing services suitable for service users by delivering accurate information related to conditions of service users and blocking many people positioned in drive-through clinics, such as medical staffs, from a risk of infection.

The methods and apparatuses of the present invention have other features and advantages which will be apparent from or are set forth in more detail in the accompanying drawings, which are incorporated herein, and the following Detailed Description, which together serve to explain certain principles of the present invention.

### BRIEF DESCRIPTION OF THE DRAWINGS

FIG. 1 partially illustrates a drive-through treatment system according to an exemplary embodiment of the present invention.

FIG. 2 illustrates a configuration of a monitoring device according to an exemplary embodiment of the present invention.

FIG. 3 illustrates an example in which facial image data is converted into facial feature data according to an exemplary embodiment of the present invention.

FIG. 4 illustrates a portion of a central management device according to an exemplary embodiment of the present invention.

FIG. 5 schematically illustrates a data augmenter according to an exemplary embodiment of the present invention.

FIG. 6 illustrates a flowchart showing a method of determining a state level according to an exemplary embodiment of the present invention.

FIG. 7 illustrates a flowchart showing a drive-through treatment method according to an exemplary embodiment of the present invention.

It may be understood that the appended drawings are not necessarily to scale, presenting a somewhat simplified representation of various features illustrative of the basic principles of the present invention. The specific design features of the present invention as disclosed herein, including, for example, specific dimensions, orientations, locations, and shapes will be determined in part by the particularly intended application and use environment.

In the figures, reference numbers refer to the same or equivalent parts of the present invention throughout the several figures of the drawing.

### DETAILED DESCRIPTION

Reference will now be made in detail to various embodiments of the present invention(s), examples of which are illustrated in the accompanying drawings and described below. While the present invention(s) will be described in conjunction with exemplary embodiments of the present invention, it will be understood that the present description is not intended to limit the present invention(s) to those exemplary embodiments. On the other hand, the present invention(s) is/are intended to cover not only the exemplary embodiments of the present invention, but also various alternatives, modifications, equivalents and other embodiments, which may be included within the spirit and scope of the present invention as defined by the appended claims.

The present invention can perform various information collection and examination or treatment based on communication or network and platform linkage establishment. Flexibility of information delivery may be secured and data transmission may be minimized by selecting an optimal data format for service user information and applying various vehicle communication or data techniques to service user information delivery.

Exemplary embodiments of the present application will be described more fully hereinafter with reference to the accompanying drawings, in which embodiments of the present invention are shown. As those skilled in the art would realize, the described embodiments may be modified in various different ways, all without departing from the spirit or scope of the present invention.

It is to be understood that when one component is referred to as being “connected” or “coupled” to another component, it may be connected or coupled directly to the other component or be connected or coupled to the other component with a further component intervening therebetween. On the other hand, it is to be understood that when one component is referred to as being “connected or coupled directly” to another component, it may be connected to or coupled to the other component without another component intervening therebetween.

It will be further understood that terms “comprises/includes” or “have” used in the present specification specify the presence of stated features, numerals, steps, operations, components, parts, or a combination thereof, but do not preclude the presence or addition of one or more other features, numerals, steps, operations, components, parts, or a combination thereof.

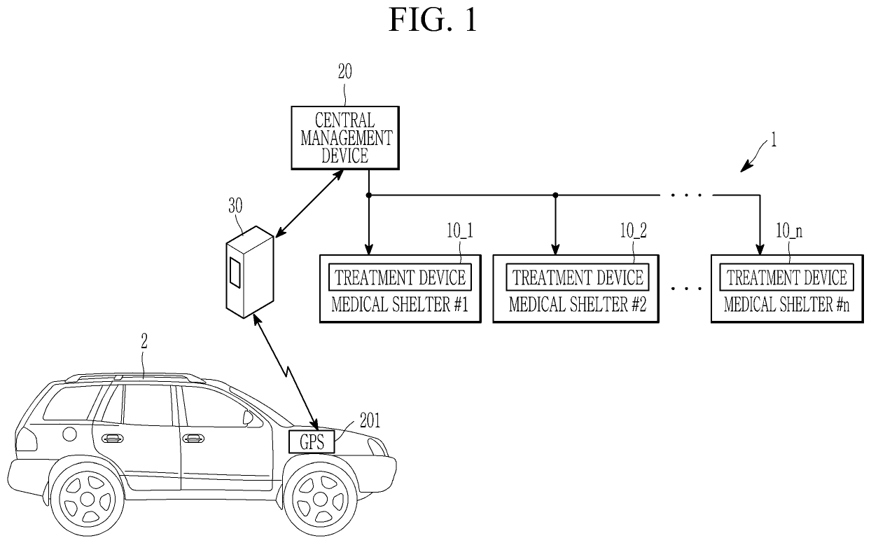

FIG. 1 partially illustrates a drive-through treatment system according to an exemplary embodiment of the present invention.

In the drive-through treatment system 1, a plurality of medical shelters #1 to #n are located, and it may be applied to a drive-through clinic in which a medical subject in a vehicle receives medical treatment while moving through the medical shelters #1 to #n by vehicle. In each of the medical shelters #1 to #n, an examination, treatment, or event (receipt, reservation, etc.) related to any one of examination and treatment is performed, and appropriate devices are provided depending on tasks performed by each medical shelter. Hereinafter, the device provided in each medical shelter is defined as a medical treatment device. When treatment is performed in a medical shelter, a doctor may be located in the medical shelter, and when an examination is performed in a medical shelter, a doctor or engineer specializing in examination may be located.

The drive-through treatment system 1 includes a plurality of medical treatment devices 10_1 to 10_n positioned at a plurality of medical shelters #1 to #n, a central management device 20, and a monitoring device 30.

As illustrated in FIG. 1, when a vehicle 2 enters a section in which the drive-through treatment system 1 is established, the monitoring device 30 may detect the vehicle 2. The monitoring device 30 may generate a plurality of body information data by detecting a plurality of pieces of body information related to a service user (e.g., a driver) who boarded the vehicle 2, and transit section data may be generated by acquiring Global Positioning System (GPS) data related to a movement path of the vehicle 2 from the vehicle 2. The monitoring device 30 transmits the plurality of body information data and the transit section data to the central management device 20, and the central management device 20 determines a condition of the service user according to the plurality of body information data and the transit section data. The central management device 20 may transmit the condition of the service user to each of the medical treatment devices 10_1 to 10_n. Each of the medical treatment devices 10_1 to 10_n may transmit information to other adjacent treatment devices or may request necessary information. The vehicle 2 may transmit information necessary for treatment to the medical treatment devices positioned in the medical shelters #1 to #n while passing through the medical shelters #1 to #n.

Information transmission and reception between the plurality of medical treatment devices 10_1 to 10_n, the central management device 20, and the monitoring device 30 illustrated in FIG. 1 may be implemented through a wireless local area network (WLAN). Furthermore, information transmission and reception between the vehicle 2, the monitoring device 30, and the medical treatment devices 10_1 to 10_n may be implemented through a wireless local area network (WLAN). However, the present invention is not limited thereto, and it may be implemented by WLAN or various communication methods other than WPAN.

The vehicle 2 includes a GPS 201, and may transmit GPS data regarding the movement path of vehicle 2 to the monitoring device 30.

Each of the medical treatment devices 10_1 to 10_n may receive and display information related to a condition of a service user from the central management device 20. Medical staffs located in the medical shelters #1 to #n may prepare necessary medical treatments, examinations, and other measures depending on the condition of the displayed service user. Each of the medical treatment devices 10_1 to 10_n includes various configurations necessary to perform measures such as treatment, examination, etc. on a service user for at least one specific subject. Each of the medical treatment devices 10_1 to 10_n is fixed at a specific position, and may process information related to a treatment result inputted from a doctor performing treatment and transmit it to the adjacent treatment device and the central management device 20. Furthermore, each of the medical treatment devices 10_1 to 10_n may display the received information related to the service user to the outside such that the received information may be used for treatment. The information related to the service user may include condition information of the service user.

When the monitoring device 30 fails to detect body information for a service user, the monitoring device 30 may notify the central management device 20 of the present failure. When the central management device 20 receives a notification of failure to detect body information, the central management device 20 notifies treatment devices 10_1 to 10_n of the failure to detect the body information. Medical staffs located in each of the medical shelters #1 to #n may recognize in advance that there is no body information on the service user through the medical treatment devices 10_1 to 10_n, and may prepare necessary measures.

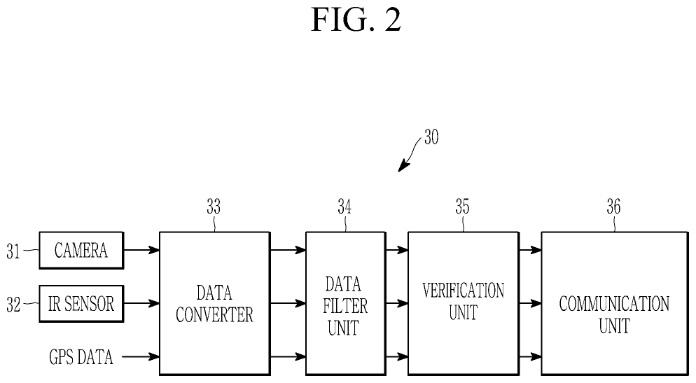

FIG. 2 illustrates a configuration of a monitoring device according to an exemplary embodiment of the present invention.

As illustrated in FIG. 2, the monitoring device 30 includes a camera 31, an infrared ray (IR) sensor 32, a data converter 33, a data filter unit 34, a verification unit 35, and a communication unit 36.

The camera 31 generates facial image data by photographing a face of the service user.

The Infrared sensor (IR sensor) 32 detects a temperature of the service user to generate temperature data.

The data converter 33 receives facial image data, temperature data, and GPS data, and converts each received data depending on a set conversion method.

The data converter 33 may determine main points for determining a facial contour and an expression based on the facial image data, and may generate facial feature data representing the main points. For example, the data converter 33 may determine main points such as eyes, nose, mouth, and cheekbones positioned within the facial contour, and may generate facial feature data representing shapes such as eyes, nose, mouth, and cheekbones.

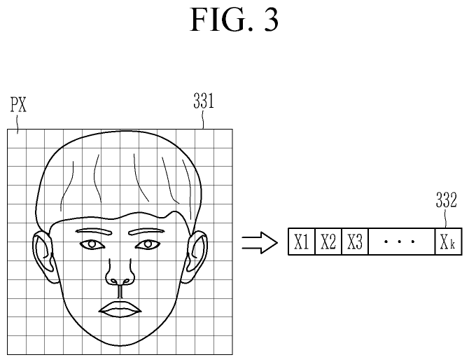

FIG. 3 illustrates an example in which facial image data is converted into facial feature data according to an exemplary embodiment of the present invention.

As illustrated in FIG. 3, facial image data 331 includes image data for each of a plurality of cells PX forming a face in a two-dimensional plane. The data converter 33 generates facial feature data 332 in which one-dimensional data representing each of main points in a facial contour is serially arranged. In FIG. 3, each of X1 to Xk indicates data representing a shape of a main point, and k may depend on a total number of main points.

The data converter 33 may generate body temperature data by converting the temperature data depending on a body temperature range of a person. For example, the data converter 33 may convert temperature data implemented as binary data indicating 0 to 255 into body temperature data (30 to 45° C.) having a predetermined range based on a normal body temperature of a person.

The data converter 33 may determine a movement path of a service user who has moved by the vehicle 2 based on GPS data received from the vehicle 2. The data converter 33 may divide a specific region into a plurality of sections, may determine sections through which the vehicle 2 passes depending on a movement path, and may generate transit section data indicating the sections through which the vehicle 2 passes depending on an elapsed time taken by the vehicle. In the instant case, the specific region is a region of a size that a service user can move by vehicle, and may be an entire territory of the Republic of Korea.

The data filter unit 34 filters data received from the data converter 33 to remove noise from the received data. Data passing through the data filter unit 34 is transmitted to the verification unit 35.

The verification unit 35 verifies the data received through the data filter unit 34 based on a matching range corresponding to each of the data received through the data filter unit 34. The verification unit 35 receives facial feature data, body temperature data, and transit section data through the data filter unit 34. When each data set deviates from a matching range corresponding to each of the facial feature data, the body temperature data, and the transit section data, the verification unit 35 may change each data set to a corresponding set value. For example, when the body temperature data includes an infinite value, the verification unit 35 may change a corresponding value to a predetermined value (e.g., a survival upper limit temperature of 45° C.). When there is an insignificant value (null) which is not normally converted in each of the facial feature data, the body temperature data, and the transit section data generated by the data converter 33, the verification unit 35 may change a null value of each of the facial feature data, the body temperature data, and the transit section data to a specific value instructing to pass a null value.

The communication unit 36 transmits the facial feature data, the body temperature data, and the transit section data to the central management device 20.

In addition to a camera and an infrared sensor (IR sensor) illustrated in FIG. 2, various sensors, such as a voice sensor configured for detecting the voice of a service user, may be added to the monitoring device 30. Sensing information generated from the various sensors forming the monitoring device 30 is parallelly passed through the data converter 33, the data filter unit 34, the verification unit 35, and the communication unit 36 to be transmitted to the central management device.

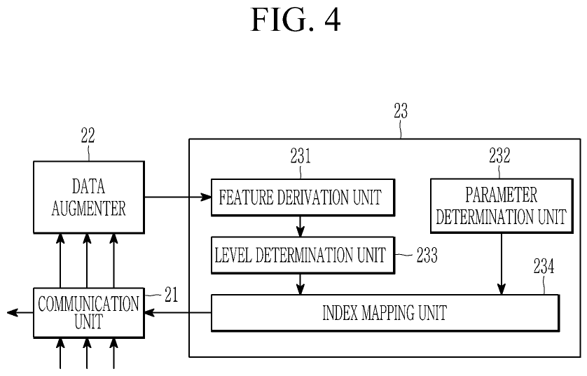

FIG. 4 illustrates a portion of a central management device according to an exemplary embodiment of the present invention.

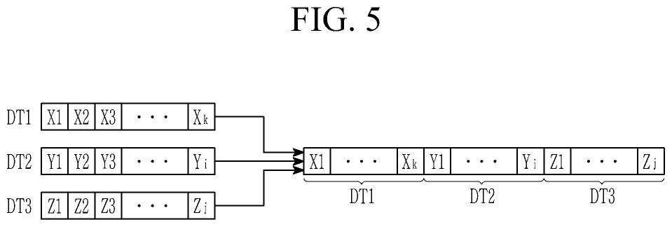

FIG. 5 schematically illustrates a data augmenter according to an exemplary embodiment of the present invention.

The central management device 20 includes a communication unit 21, a data augmenter 22, and a condition determination unit 23, and the condition determination unit 23 includes a feature derivation unit 231, a parameter determination unit 232, a level determination unit 233, and an index mapping unit 234.

The communication unit 21 receives facial feature data (X1 to Xk, DT1), body temperature data (Y1 to Yi, DT2), and transit section data (Z1 to Zj, DT3) from the communication unit 36 of the monitoring device 30 and transmits the data to the data augmenter 22.

The data augmenter 22 may augment the plurality of body information data and the transit section data to generate augmented data. The plurality of body information data includes the facial feature data DT1 and the body temperature data DT2. When augmenting the facial feature data DT1, the body temperature data DT2, and the transit section data DT3, the data augmenter 22 converts the data into a form suitable for the condition determination unit 23 to generate augmented data. The data augmenter 22 may generate augmented data by serially arranging the facial feature data DT1, the body temperature data DT2, and the transit section data DT3. In FIG. 5, the facial feature data DT1, the body temperature data DT2, and the transit section data DT3 are serially arranged in the present order, but the arrangement order may be changed.

The condition determination unit 23 may receive the augmentation data from the data augmenter 22, may determine a condition of a service user based on the augmented data, and may classify a condition level.

First, the feature derivation unit 231 analyzes the augmented data to determine a current pain degree of the service user, a body temperature of the service user, and a number of infectious disease occurrence sections that the service user passed through. For example, the feature derivation unit 231 may store a plurality of reference facial feature data, and may determine reference facial feature data which is most similar to the facial feature data of the augmented data among the plurality of reference facial feature data to determine a current pain degree of the service user depending on the determined reference facial feature data. The feature derivation unit 231 may determine the body temperature of the service user by determining an average of the body temperature data of the augmented data or by deriving a median value thereof. Furthermore, the feature derivation unit 231 may compare the transit section data of the augmented data with occurrence section data indicating a section where an infectious disease has occurred, and may determine how many infectious disease occurrence sections the service user has passed through.

The parameter determination unit 232 determines a parameter for each of a current pain degree of the service user, a body temperature of the service user, and a number of infectious disease occurrence sections that the service user has passed through. For example, the parameter may be a weight value which is multiplied by each data set to determine the condition of the service user. The parameter determination unit 232 may determine the parameter for each data set by learning previously accumulated data through a neural network. The previously accumulated data may include data related to the current pain degree of the service user, the body temperature thereof, the number of infectious disease occurrence sections that the service user passed through, and whether or not the actual infectious disease was transmitted. The parameter determination unit 232 may determine a parameter for the current pain degree of the service user, a parameter for the body temperature thereof, and a parameter for the number of infectious disease occurrence sections that the service user has passed through.

The level determination unit 233 determines a level for each of the current pain degree of the service user, the body temperature of the service user, and the number of infectious disease occurrence sections, received from the feature derivation unit 231. The level determination unit 233 may determine the current pain level as one of 0, 1, . . . , and LN1 based on the current pain degree of the service user, the body temperature level as one of 0, 1, . . . , and LN2 based on the body temperature of the service user, and the level for the number of infectious disease occurrence sections as one of 0, 1, . . . , and LN3 based on the number of infectious disease occurrence sections. Each of LN1 to LN3 is a natural number of 2 or more, and may be appropriately set depending on a quantification degree. The level determination unit 233 may increase the level of the corresponding features as the current pain degree of the service user increases, the body temperature increases, and the number of infectious disease occurrence sections increases. When the service user passes through an infectious disease occurrence section, the level determination unit 233 may increase the level as the number of infectious disease occurrence sections increases. The quantified current pain level is called a pain level, the quantified body temperature level is called a body temperature level, and the quantified level of the number of infectious disease occurrence sections is called an infection risk level.

The index mapping unit 234 may receive the pain level, the body temperature level, and the infection risk level from the level determination unit 233, may receive the parameters for the current pain degree of the service user, the body temperature thereof, and the number of infectious disease occurrence sections from the parameter determination unit 232, may determine a value multiplied by a parameter corresponding to each of the pain level, body temperature level, and infection risk level, and may determine an index indicating the condition of the service user by adding the determined multiplied values. The index mapping unit 234 may determine a condition level (e.g., one of levels 0 to 4) indicating the condition of the service user based on the index.

The communication unit 21 may receive the condition level from the index mapping unit 234, and may transmit it to the plurality of medical treatment devices 10_1 to 10_n.

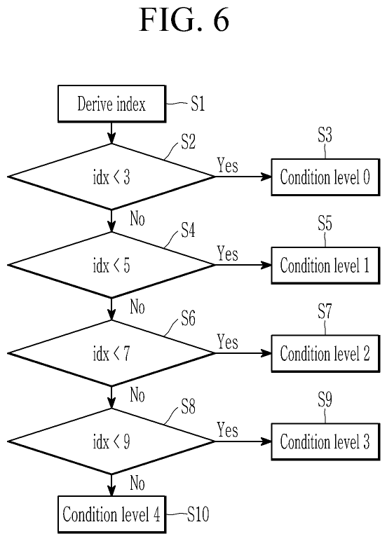

FIG. 6 illustrates a flowchart showing a method of determining a state level according to an exemplary embodiment of the present invention.

The index mapping unit 234 may set reference values to be compared with the index depending on a condition in which the parameter for the body temperature is 2, the parameter for the current pain level is 1, and the parameter for the number of infectious disease occurrence sections is 1.

The index mapping unit 234 may determine an index idx by adding a value obtained by multiplying the body temperature level by the parameter 2, a value obtained by multiplying the pain level by the parameter 1, and a value obtained by multiplying the infection risk level by the parameter 1 (S1).

The index mapping unit 234 may compare the determined index idx with a first reference value “3” (S2). As a result of the comparison in step S2, when the index idx is smaller than 2, the index mapping unit 234 determines the condition of the service user as a condition level 0 (condition level 0 is a stable state) (S3).

The index mapping unit 234 may compare the determined index idx with a second reference value “5” (S4). As a result of the comparison in step S4, when the index idx is smaller than 5, the index mapping unit 234 determines the condition of the service user as a condition level 1 (condition level 1 is a cautious but stable state) (S5).

The index mapping unit 234 may compare the determined index idx with a third reference value “7” (S6). As a result of the comparison in step S6, when the index idx is smaller than 7, the index mapping unit 234 determines the condition of the service user as a condition level 2 (condition level 2 is a cautious and unstable state (S7).

The index mapping unit 234 may compare the determined index idx with a fourth reference value “9” (S8). As a result of the comparison in step S8, when the index idx is smaller than 9, the index mapping unit 234 determines the condition of the service user as a condition level 3 (condition level 3 is an urgent and unstable state (S9).

As a result of the comparison in step S8, when the index idx is equal to or greater than 9, the index mapping unit 234 determines the condition of the service user as a condition level 4 (condition level 4 is an emergency and unstable state (S10).

The index mapping unit 234 determines a condition level indicating the condition of the service user, and transmits the condition level to the communication unit 21. The communication unit 21 transmits the condition level to the medical treatment devices 10_1 to 10_n.

In step S1, the index mapping unit 234 determines an index based on the current pain degree, the body temperature, and the number of infectious disease occurrence sections that the service user has passed through, but the present invention is not limited thereto. For example, the index mapping unit 234 may determine the index by use of two of the current pain degree, the body temperature, and the number of infectious disease occurrence sections. In the instant case, the reference values may be appropriately set depending on the characteristic reflected in the index derivation and a parameter value corresponding thereto.

Hereinafter, a drive-through treatment method according to an exemplary embodiment will be described.

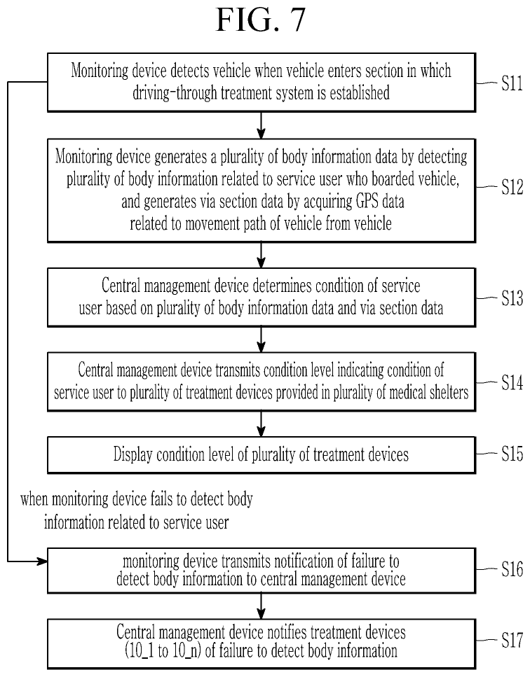

FIG. 7 illustrates a flowchart showing a drive-through treatment method according to an exemplary embodiment of the present invention.

As illustrated in FIG. 7, when a vehicle 2 enters a section in which the drive-through treatment system 1 is established, the monitoring device 30 may detect the vehicle 2 (S11).

The monitoring device 30 may generate a plurality of body information data by detecting a plurality of body information related to a service user who boarded the vehicle 2, and may generate transit section data by acquiring GPS data related to a movement path of the vehicle 2 from the vehicle 2 (S12). The monitoring device 30 may transmit a plurality of body information data and transit section data to the central management device 20.

The central management device 20 may receive the plurality of body information data and the transit section data, and may determine a condition of a service user based on the plurality of body information data and the transit section data (S13). A method for the central management device 20 to determine the condition of the service user is the same as in the above-described embodiment. The condition of the service user may be indicated as a condition level.

The central management device 20 may transmit a condition level indicating the condition of the service user to the plurality of medical treatment devices 10_1 to 10_n provided in the plurality of medical shelters #1 to #n (S14).

Each of the medical treatment devices 10_1 to 10_n is provided with a display device, and the condition level received from the central management device 20 may be displayed on the display device by the medical treatment devices 10_1 to 10_n (S15). This allows doctors located in each shelter to prepare necessary actions in advance depending on a condition level thereof.

In step S12, when the monitoring device 30 fails to detect body information related to the service user, a notification of failure to detect the body information may be transmitted to the central management device 20 (S16).

When the central management device 20 receives a notification of failure to detect body information, the central management device 20 notifies treatment devices 10_1 to 10_n of the failure to detect the body information (S17). Medical staffs located in each of the medical shelters #1 to #n may recognize in advance that there is no body information on the service user through the medical treatment devices 10_1 to 10_n, and may prepare necessary measures.

For convenience in explanation and accurate definition in the appended claims, the terms “upper”, “lower”, “inner”, “outer”, “up”, “down”, “upwards”, “downwards”, “front”, “rear”, “back”, “inside”, “outside”, “inwardly”, “outwardly”, “interior”, “exterior”, “internal”, “external”, “forwards”, and “backwards” are used to describe features of the exemplary embodiments with reference to the positions of such features as displayed in the figures. It will be further understood that the term “connect” or its derivatives refer both to direct and indirect connection.

The foregoing descriptions of specific exemplary embodiments of the present invention have been presented for purposes of illustration and description. They are not intended to be exhaustive or to limit the present invention to the precise forms disclosed, and obviously many modifications and variations are possible in light of the above teachings. The exemplary embodiments were chosen and described to explain certain principles of the present invention and their practical application, to enable others skilled in the art to make and utilize various exemplary embodiments of the present invention, as well as various alternatives and modifications thereof. It is intended that the scope of the present invention be defined by the Claims appended hereto and their equivalents.

What is claimed is:

1. A drive-through treatment system comprising:
a monitoring device configured to generate a body information data by detecting body information related to a service user in a vehicle, and to generate transit section data related to a movement path of the vehicle from the vehicle when the vehicle enters a section where a drive-through treatment system is established; and
a central management device configured to receive the body information data and the transit section data from the monitoring device, and to determine a condition of the service user according to the body information data and the transit section data.

2. The drive-through treatment system of claim 1, further including
medical treatment devices provided in a plurality of medical shelters,
wherein the central management device transmits the condition of the service user to the medical treatment devices.

3. The drive-through treatment system of claim 1, wherein the monitoring device includes:
a camera configured to generate facial image data by photographing a face of the service user;
an infrared sensor (IR sensor) configured to detect a temperature of the service user to generate temperature data;
a data converter electrically connected to the camera and the IR sensor and configured to receive the facial image data, the temperature data, and the transit section data, and to convert each of the received facial image data, temperature data and transit section data depending on a predetermined conversion method to data;
a data filter unit configured to receive and filter the data received from the data converter to remove noise from the received data; and
a verification unit configured to verify data received through the data filter unit based on a matching range corresponding to each of the data received through the data filter unit.

4. The drive-through treatment system of claim 3, wherein the data converter is configured to determine a plurality of points for determining a facial contour and an expression based on the facial image data, and to generate facial feature data representing the plurality of points.

5. The drive-through treatment system of claim 3, wherein the data converter is configured to generate body temperature data by converting the temperature data depending on a body temperature range of a person.

6. The drive-through treatment system of claim 3, wherein the data converter is configured to determine a movement path of the service user who has moved by the vehicle according to Global Positioning System (GPS) data, to determine sections through which the vehicle passes depending on the movement path, and to generate the transit section data indicating the sections through which the vehicle passes depending on an elapsed time taken by the vehicle.

7. The drive-through treatment system of claim 3,
wherein the data received through the data filter unit includes the facial feature data, the body temperature data, and the transit section data, and
wherein the verification unit is configured to change each of the received data to a corresponding set value when each of the received data deviates from a matching range corresponding to each of the facial feature data, the body temperature data, and the transit section data.

8. The drive-through treatment system of claim 1, wherein the central management device includes:
a data augmenter configured to augment the body information data and the transit section data to generate augmented data; and
a condition determination unit configured to receive the augmentation data from the data augmenter, and to determine a condition level by determining the condition of the service user according to the augmentation data.

9. The drive-through treatment system of claim 8, wherein the data augmenter is configured to convert the body information data and the transit section data into input data in a form suitable for the condition determination unit.

10. The drive-through treatment system of claim 8,
wherein the body information data includes facial feature data and body temperature data, and
wherein the data augmenter is configured to generate the augmented data by serially arranging the facial feature data, the body temperature data, and the transit section data.

11. The drive-through treatment system of claim 8, wherein the condition determination unit includes:
a feature derivation unit configured to analyze the augmented data to determine a current pain degree of the service user, a body temperature of the service user, and a number of infectious disease occurrence sections that the service user has passed through;
a parameter determination unit configured to determine parameters for the current pain degree of the service user, the body temperature of the service user, and the number of infectious disease occurrence sections that the service user has passed through;
a level determination unit configured to determine a pain level, a body temperature level, and an infection risk level for the current pain degree of the service user, the body temperature of the service user, and the infectious disease occurrence sections that the service user has passed through; and
an index mapping unit configured to determine a value obtained by multiplying each of the pain level, the body temperature level, and the infection risk level by a corresponding one of the parameters, and determine an index indicating the condition of the service user by adding the determined multiplied values.

12. The drive-through treatment system of claim 11, wherein the index mapping unit is configured to compare the index with a plurality of reference values, and to determine the condition level indicating the condition of the service user according to a result of the comparing.

13. The drive-through treatment system of claim 12, wherein the index mapping unit is configured to:
determine the condition level as a value indicating a stable state when the index is smaller than a first reference,
determine the condition level as a value indicating a stable state that requires attention when the index is greater than or equal to the first reference value and smaller than a second reference value,
determine the condition level as a value indicating an unstable state that requires attention when the index is greater than or equal to the second reference value and smaller than a third reference value,
determine the condition level as a value indicating an urgent unstable state when the index is greater than or equal to the third reference value and smaller than a fourth reference value, and
determine the condition level as a value indicating an emergency unstable condition when the index is greater than or equal to the fourth reference value.

14. The drive-through treatment system of claim 12, further including medical treatment devices provided in a plurality of medical shelters,
wherein the central management device transmits the condition level to the medical treatment devices.

15. The drive-through treatment system of claim 14, wherein each of the medical treatment devices includes a display device and is configured to display the condition level received from the central management device through the display device.

16. The drive-through treatment system of claim 15,
wherein the monitoring device is configured to transmit a notification of failure to detect the body information to the central management device when the monitoring device fails to detect the body information related to the service user; and
wherein the central management device is configured to notify the medical treatment devices provided in the medical shelters of the failure to detect the body information when the central management device receives the notification of the failure to detect the body information from the monitoring device.

17. A drive-through treatment method comprising:
detecting, by a monitoring device, a vehicle when the vehicle enters a section where a drive-through treatment system is established;
generating, by the monitoring device, a body information data by detecting body information related to a service user in the vehicle, and generating transit section data by acquiring Global Positioning System (GPS) data related to a movement path of the vehicle from the vehicle; and
receiving, by a central management device, the body information data and the transit section data from the monitoring device, and determining a condition of the service user according to the body information data and the transit section data.

18. The drive-through treatment method of claim 17, further including
transmitting, by the central management device, a condition level indicating the condition of the service user to a plurality of medical treatment devices provided in a plurality of medical shelters.

19. The drive-through treatment method of claim 18, further including
displaying, by each of the medical treatment devices, the condition level received from the central management device through a display device.

20. The drive-through treatment method of claim 18, further including:
transmitting, by the monitoring device, a notification of failure to detect the body information to the central management device when the monitoring device fails to detect the body information related to the service user; and
notifying, by the central management device, the medical treatment devices provided in medical shelters of the failure to detect the body information when the central management device receives the notification of the failure to detect the body information from the monitoring device.
